(19) United States
(12) Patent Application Publication
(10) Pub. No.: US 2002/0038465 A1
(43) Pub. Date: Mar. 28, 2002

(54) Non-human animal mutants and methods for production of the same

(76) Inventors: Moriaki Kusakabe, Ibaraki (JP); Yasushige Yano, Saitama (JP)

Correspondence Address: Foley & Lardner, Washington Harbour, 3000 K Street N W, Suite 500, Washington, DC, 20007-5109, US

(21) Appl. No.: 09/539,526

(22) Filed: Mar. 30, 2000

(30) Foreign Application Priority Data
Sep. 30, 1999 (JP) 279324/1999

(51) Int. Cl. A01K 67/00; A61D 7/00

(52) U.S. Cl. 800/8; 800/9; 435/446; 424/561; 600/33

(57) ABSTRACT

This invention relates to a method for producing non-human animal mutants, characterized in that the method comprises irradiating a male germ cell with heavy ion beams and selecting a mutated animal from animals, which are produced from an egg and the irradiated male germ cell, and their generations. According to the invention, non-human animal mutants with various phenotypes or morphological characters can efficiently be produced.

### FIELD OF THE INVENTION

The present invention relates to non-human animal mutants and methods for producing the same. According to the invention non-human animal mutants with various phenotypes or morphological characters are efficiently producible.

### BACKGROUND OF THE INVENTION

It is well known that animal mutants are useful for analysis of gene functions, and some chemicals including ENU, which are a type capable of inducing mutation of a single nucleotide in DNA molecule, have mainly been used for producing the animal mutants so far. As there is little lethal effect of the mutation on animals, efficient production of animal mutants can be conducted. However, as this method mainly makes the mutation of a single nucleotide, it is rare that the mutation of a single nucleotide gives dramatic change in its phenotype. Moreover, as it is hard to detect the mutation of a single nucleotide, this method is inconvenience in examining the relationship between a mutated gene and its phenotype.

Another chemical such as chlorambucil enables producing a so-called deletion mutant, in which a certain region of DNA is deleted. As such mutagenesis often gives lethal effect on animals, this type of chemical is considered to be inadequate for efficient production of a mutant, but it often gives considerable change in a phenotype of the mutant produced. Therefore, the chemical like chlorambucil is often preferred to the chemical like ENU in producing the mutant valuable for analysis of gene functions.

It is believed that mutagenesis with chemical substances occurs in a certain limited region of DNA. For analysis of a number of gene functions, however, it is desirable that mutagenesis occurs in extensive regions of DNA, not in a limited region of DNA. Consequently, development of a new technology for animal mutants, in which a mutagenesis occurs in extensive regions in their DNA, has been desired.

Besides the chemical treatment, use of heavy ion beams was proposed to produce mutants in plant. For instance, Nakai et al. (RIKEN Accel. Plog Rep. 26, 109, 1992) reported their success in producing a new rice plant resistant to bacterial leaf blight by irradiation of heavy ion beams to rice seeds. Abe et al. (Japanese Patent Application Laid-Open (Kokai) No. 9-28220) reported that irradiation of heavy ion beams to a pollinated tobacco ovary produced a new chlorophyl deficient tobacco plant with morphological anomalies such as leaf in needle shape, less sensitivity for gravity, etc.

As it is believed that mutagenesis with heavy ion beams occurs in extensive regions of DNA not in a certain limited region of DNA, it may readily become practicable to produce animal mutants with various phenotypes by irradiation of heavy ion beams if the beams cause mutagenesis in animals as seen in plants.

In application of the heavy-ion-beam irradiation for producing animal mutants, however, there are some problems. The first problem is animal's lower tolerance against radiation as compared with plants, therefore irradiation of heavy ion beams having severe injury effect on genes will probably prevent normal growth of embryo. The second problem is that differentiation of an animal germ cell occurs more rapidly than that of plant, so maintenance of animal mutants as strain will probably be difficult because of deficiency of the germ cell even if the embryo itself makes normal growth.

Due to the above reasons, success in producing animal mutants by means of heavy ion beams irradiation has not yet been reported.

The present invention was accomplished under the technical background described above, and its object is to provide new means for producing non-human animal mutants by irradiation of heavy ion beams. After intensive studies to solve the aforesaid problems the present inventor has now found that various mutations are produced with high frequency in animals, which are produced from a normal egg and a male germ cell such as spermatozoon irradiated with heavy ion beams, and thereby completed this invention.

### SUMMARY OF THE INVENTION

The present invention is directed to a method for producing the non-human animal mutants, characterized in that the method comprises irradiating a male germ cell with heavy ion beams and selecting a mutated animal from animals, which are produced from a normal egg and the irradiated male germ cell, and their generations.

The present invention is further directed to a non-human animal mutant produced by the method above.

This specification includes part or all of the contents as disclosed in the specification and/or drawings of Japanese Patent Application No.279324/1999, which is a priority document of the present application.

### BRIEF DESCRIPTION OF THE DRAWINGS

FIG. 1 shows the photographs of mouse mutants derived from spermatozoa irradiated with heavy ion beams.

FIG. 2 shows the graphs depicting the relationship between various modes of irradiation and % regeneration of seminiferous tubule epithelial germ cells.

FIG. 3 shows the photographs showing testis tissues of mice irradiated with heavy ion beams.

FIG. 4 shows the photographs of embryos of G1 mice with morphological anomalies.

FIG. 5 shows the photographs of embryos of G1 mouse with morphological anomaly.

FIG. 6 shows the photographs of embryos of G3 mice with morphological anomalies.

### DETAILED DESCRIPTION OF THE INVENTION

The method for producing animal mutants according to this invention is characterized by irradiating a male germ cell with heavy ion beams and then selecting a mutated animal from animals, which are produced from a normal egg and the irradiated male germ cell, and their generations (i.e., descendants or offspring).

As the male germ cell, postmeiotic spermatozoa and premeiotic spermatogonium can be used. In use of spermatogonium, the testis of a male animal is irradiated with heavy ion beams, then the male animal is crossed with a female animal to produce animals in the first generation. Selection of animal mutants is conducted among the animals in the first and successive generations. In use of spermatozoa, spermatozoa collected from the testis of a male animal are irradiated with heavy ion beams, and subsequently the spermatozoa are used for artificial fertilization with a non-fertilized egg in vitro, and the fertilized egg is implanted in a pseudopregnant mother to produce animals in the first generation. Selection of animal mutants is conducted among the animals in the first and successive generations. There are some drawbacks in the use of spermatogonium. That is, the beam dose has a limitation because much dose may give lethal damage to the male animal itself. Additionally, in the method using spermatogonium wherein fertilization has to be conducted by crossing not artificial fertilization, animal mutants can not be obtained if spermatozoa with mutation can not reach an egg due to competition with normal spermatozoa. This kind of problem, however, does not occur in the method using spermatozoa. Thus, spermatozoa is preferable to spermatogonium as the male germ cell.

There is no limitation for the kind of heavy ion beam as far as it can induce mutation in animals, and examples of the usable heavy ion beam include nitrogen (N) ion beam, carbon (C) ion beam, neon (Ne) ion beam, argon (Ar) ion beam, and iron (Fe) ion beam. Dose of heavy ion beams can be determined depending on kinds of male germ cells. In the case of irradiation to spermatogonium, i.e. male animal testis, irradiation dose exceeding 5 Gy brings severe damage to the cell resulting in extremely low pregnancy chance, so the preferred dose is 5 Gy or less, more preferably around 3 Gy. In the case of irradiation to spermatozoa collected from the testis, dose exceeding 50 Gy brings severe damage to the spermatozoa, and the irradiation of a dose less than 10 Gy results in low probability of mutagenesis. Dose range of irradiation, therefore, is preferably 10-50 Gy, more preferably 30-50 Gy.

Any animal except human can be used for producing animal mutants of interest, and mammals, more particularly mouse and rat, are preferred for the use.

Egg to be used for crossing with male germ cell is not limited to special one but it is preferably normal one without exposure to heavy ion beams. In the first generation animal (G1 animal) produced by fertilization of a normal non-fertilized egg and a spermatozoon treated with heavy ion beams, mutation in the spermatozoon does not appear as its phenotype if the mutation is inherited recessively. Even in this case, an animal with recessive mutation can be obtained by examining phenotypes of successive generations of the G1 animal. The procedure specifically comprises: crossing the G1 animal with a normal animal to produce G2 animals; backcrossing the G2 animal with the G1 animal to produce G3 animals; and selecting an animal with recessive mutation from G3 animals wherein any recessive mutation appears as a phenotype.

The non-human animal mutant thus obtained can be utilized as a tool to produce an animal model for studying human diseases and also to analyze a specified gene function.

### EXAMPLES

The present invention will be illustrated in more detail referring to the following examples, but it is construed that the scope of the invention is not limited to the examples.

### Example 1

Mature male mice (C57BL/6J strain, purchased from Japan Kurea K. K., Shizuoka, Japan) were sacrificed by cervical dislocation after anesthesia, and the cauda epididymides were removed from the testes. Spermatozoa were collected from the cauda epididymides with a fine needle, suspended in 1.5 ml centrifuge tubes containing 1 ml of M2 medium, whose composition is shown in Table 1, and stored at 4° C. until irradiation of heavy ion beams. The resulting suspensions were centrifuged briefly at room temperature, and were exposed to five kinds of heavy ion beams (⁴⁰Ar at 95 MeV/u, ^{5 6}Fe at 90 MeV/u, ^{2 0}Ne at 135 MeV/u, ^{1 4}N at 135 MeV/u, and C at 135MeV/u) with dose ranges of 5, 10, 20, 30, 50, 75, and 100 Gy at Riken Ring Cyclotron Facility (Wako-shi, Saitama, Japan). After irradiation, the spermatozoa were stored at 4° C. or in liquid nitrogen.

TABLE 1
Composition of M2 medium

| Component | g/L |
|---|---|
| NaCl | 5.533 |
| KCl | 0.356 |
| CaCl₂.2H₂O | 0.252 |
| KH₂PO₄ | 0.162 |
| MgSO₄.7H₂O | 0.293 |
| NaHCO₃ | 0.349 |
| HEPES | 4.969 |
| Sodium lactate | 2.610 |
| Sodium pyruvate | 0.036 |
| Glucose | 1.000 |
| BSA | 4.000 |
| Penicillin G-potassium | 0.060 |
| Streptomycin sulfate | 0.050 |
| Phenolred | 0.010 |
| Distilled water | 1 L |

On the other side, non-fertilized mouse eggs were collected from oviducts after treating female mice with pregnant mare's serum hormone and human chorionic gonadotropin. The eggs were held with a holding pipette, a single spermatozoon was picked up by micropipette and injected into the egg cytoplasm. Manipulated eggs were incubated in M16 medium, whose composition is shown in Table 2, overnight, then successfully cleaved 2-cell embryos were implanted in oviducts of pseudopregnant mothers to produce GI mice.

TABLE 2
Composition of M16 medium

| Component | g/L |
|---|---|
| NaCl | 5.533 |
| KCl | 0.356 |
| CaCl₂.2H₂O | 0.252 |
| KH₂PO₄ | 0.162 |
| MgSO₄.7H₂O | 0.293 |
| NaHCO₃ | 2.101 |
| Sodium lactate | 2.610 |
| Sodium pyruvate | 0.036 |
| Glucose | 1.000 |
| BSA | 4.000 |
| Penicillin G-potassium | 0.060 |
| Streptomycin sulfate | 0.050 |
| Phenolred | 0.010 |
| Distilled water | 1 L |

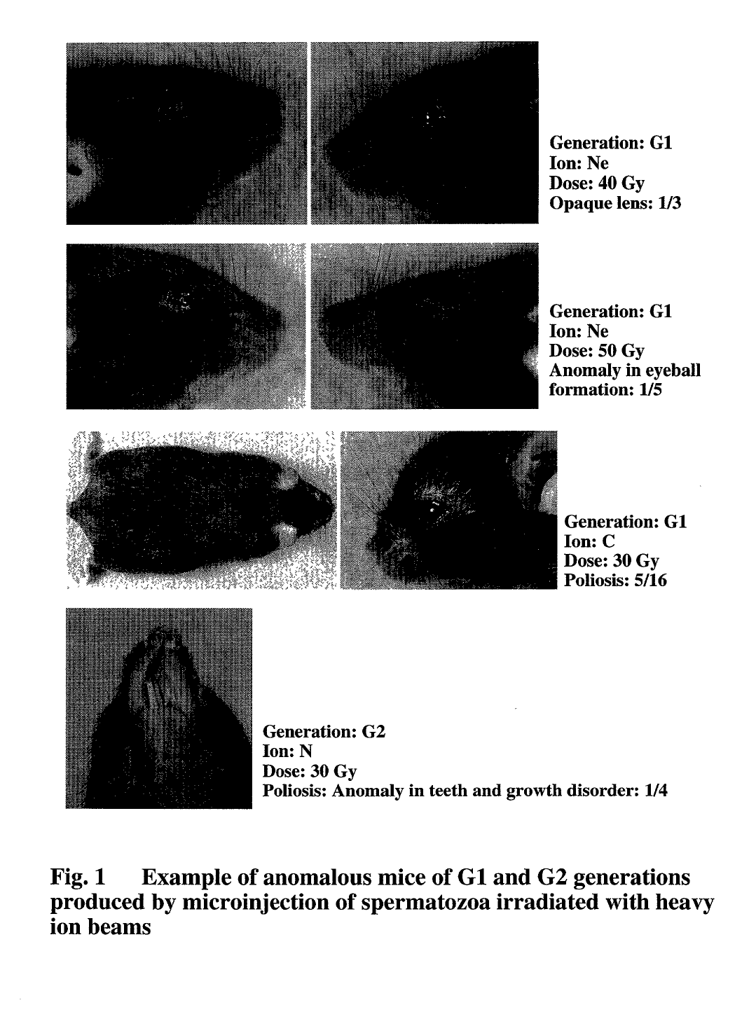

Of 1242 non-fertilized eggs into which spermatozoa had been injected, 483 eggs were cleaved into 2-cell embryos (38.9%), and 83 (17.2%) G1 mice were obtained, where some mutations were found in 8 mice (9.6%). The 8 G1 mice were crossed with a wild type C57BL/6J strain, and 444 G2 mice were produced. The G2 mice were backcrossed with the G1 mice to produce 97 G3 mice. Table 3 shows principal mutations observed in the generations ranging from G1 to G3, and FIG. 1 shows photographs of mice with anomalies.

TABLE 3

| | | | Affected tissue/ | | No. |
|---|---|---|---|---|---|
| Beams | Dose | Gen. | organ/system | Anomaly | of mice |
| ¹²C | 30 | G1 | coat color | non-pigmented hair around eyes and | 2 |
| | | | | on the back | |
| ¹⁴N | 10 | G1 | eye | cataract | 1 |
| ¹⁴N | 50 | G1 | reproduction | sterile female | 1 |
| ²⁰Ne | 10 | G1 | skin, eye | dermatitis and corneal inflammation | 1 |
| ²⁰Ne | 10 | G1 | reproduction | sterile male | 3 |
| ²⁰Ne | 10 | G1 | hair, eye | complete loss of hair | 1 |
| | | | | microphthalmia | |
| ²⁰N | 50 | G1 | hair, skin, eye | complete loss of hair, dermatitis | 1 |
| | | | | and cataract | |
| ²⁰Ne | 50 | G1 | tail | round tail tip | 1 |
| ²⁰Ne | 50 | G1 | eye | cataract and anophthalmia | 2 |
| ¹²C | 30 | G2 | coat color | many non-pigmented hairs | 2 |
| ¹²C | 30 | G2 | hair | loss of hair | 1 |
| ¹²C | 30 | G2 | hair, eye | microphthalmia and hair loss | 1 |
| ⁵⁶Fe | 75 | G2 | tooth | long teeth | 1 |
| ¹⁴N | 50 | G2 | eye | cataract | 1 |
| ¹⁴N | 50 | G2 | tooth | long teeth | 4 |
| ¹⁴N | 50 | G2 | reproduction | veginal atresia | 1 |
| ¹⁴N | 50 | G2 | tail | kinky | 1 |
| ¹⁴N | 30 | G3 | brain, eye | hydrocephalus and anophthalmia | 1 |
| ¹⁴N | 50 | G3 | skin | hypopigmentation until 10 days of age | 1 |
| ²⁰Ne | 20 | G3 | tooth | long teeth | 2 |
| ²⁰Ne | 20 | G3 | eye | microphthalmia | 1 |
| ²⁰Ne | 20 | G3 | skeleton | abnormal shape of vertebrate | 1 |

### Example 2

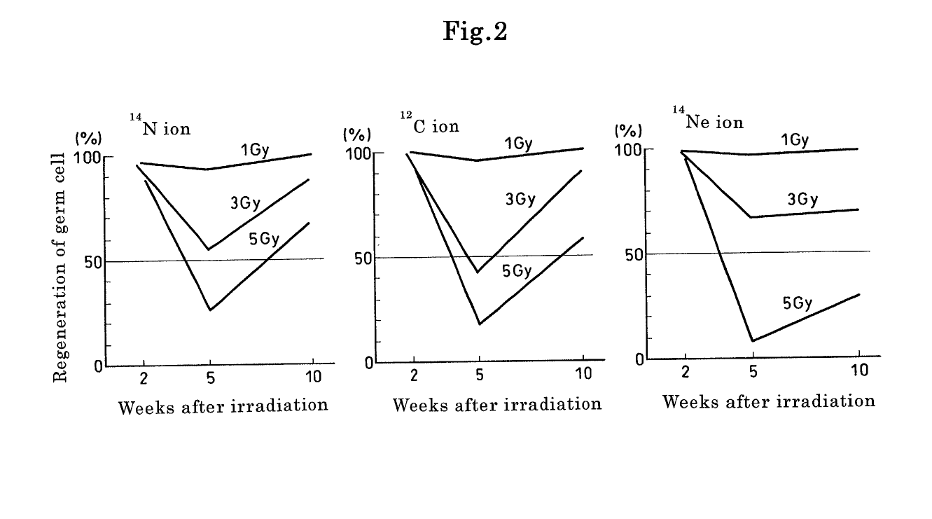

Five kinds of heavy ion beams (^{4 0}Ar (95MeV/u), ^{5 6}Fe (90MeV/u),^{2 0}Ne (135MeV/u), ^{1 4}N (135MeV/u), and ^{1 2}C (135MeV/u)) were irradiated to hypogastrium of anesthesized mature male mice with dose ranges of 0.5, 1, 3 and 5 Gy. After the irradiation, numbers of germ cells in the seminiferous epithelium were counted, and % regeneration was determined (FIG. 2). As shown in FIG. 2, deciduation of the germ cells rapidly occurred in 2 to 5 weeks after the irradiation of heavy ion beams, and new germ cells were regenerated in 5 to 10 weeks after the irradiation; probability of mutagenesis in the generated germ cells was high. The % regeneration of the germ cell was calculated from the equation below using TRA 369 antibody, which can specifically recognize germ cells from pachytene stage spermatocyte to round spermatid cell among all testis germ cells.

$$\% Regeneration of germ cell = \frac{\begin{matrix}( Number of seminiferous tubule cross section \\ containing TRA 369 antibody positive cells )\end{matrix}}{\begin{matrix}( Number of seminiferous tubule cross \\ section per testis cross section )\end{matrix}} \times 100$$

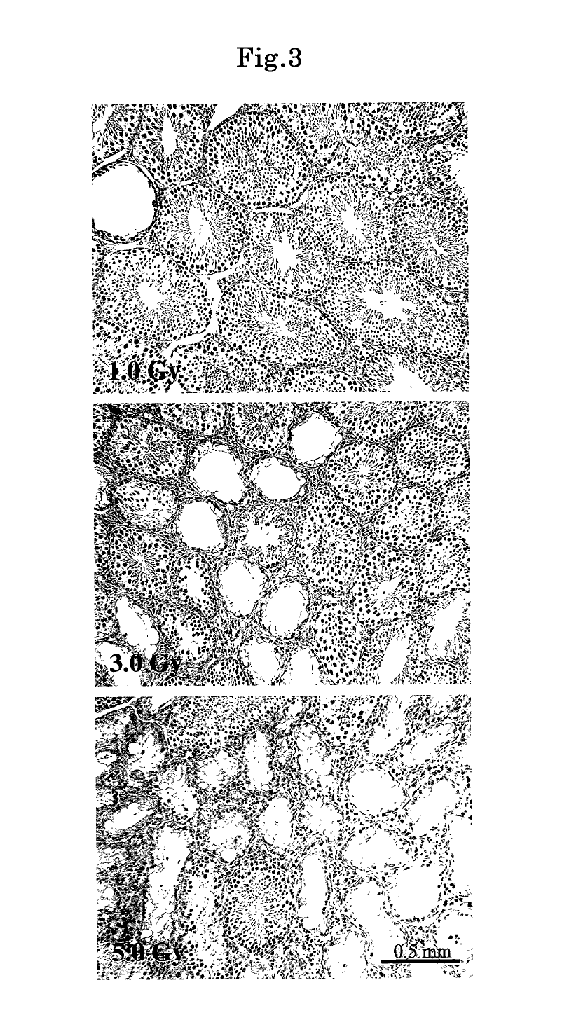

FIG. 3 shows photographs of mouse testis tissue at the time point of 10 weeks after irradiation of neon ion beams. As shown in this figure, 5-Gy irradiation caused severe tissue injury or damage to render the male mouse infertile, while 1-Gy irradiation caused only a little injury on the germ cell. Irradiation of 3Gy also caused severe tissue injury, but the germ cell in turn began to proliferate and restored an ability to fertilize. Consequently, where mutation is induced by irradiating a mouse testis with heavy ion beams, irradiation of around 3 Gy is likely to be practical.

### Example 3

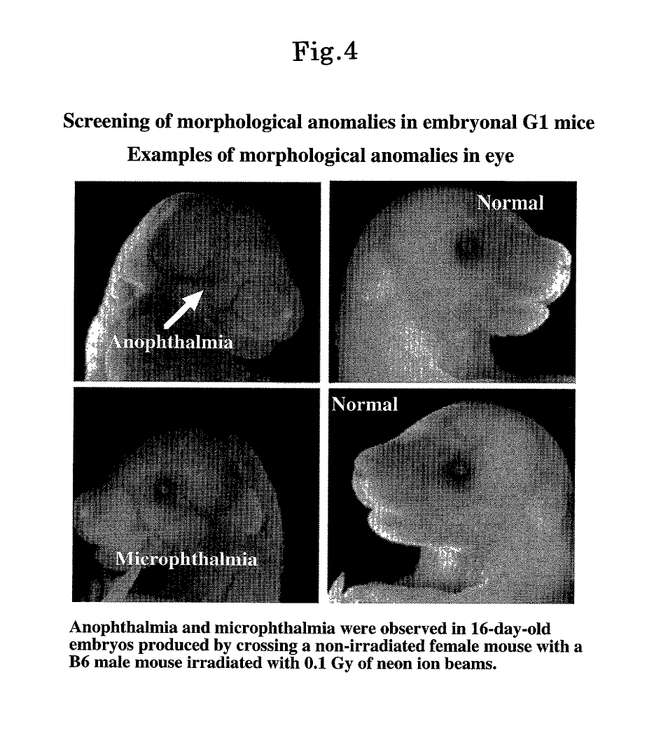

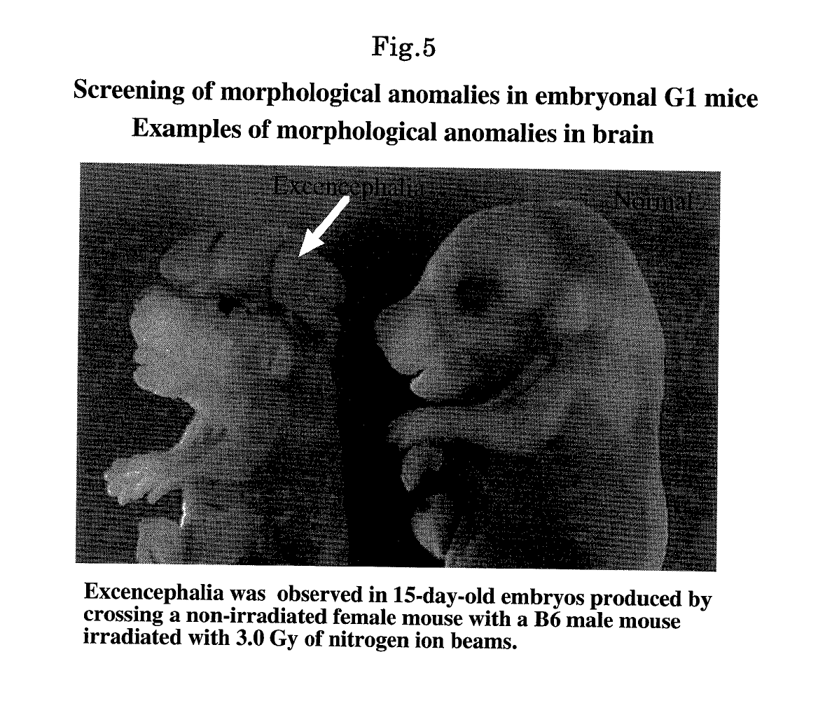

Male mice irradiated with heavy ion beams as in Example 2 were crossed with normal female mice to observe the state of embryos produced in the pregnant female mice. Crossing was conducted in 175 pairs, 146 female mice of which were impregnated and implantation of 1192 embryos was confirmed. Of these embryos, 123 embryos showed morphological anomalies as seen in FIGS. 4 and 5, and some were absorbed in the process of their growth (mutation, 10.3%). FIG. 4 is photographs of 16-day-old embryos whose male parent was irradiated with 0.1 Gy of neon ion beams, showing anomalies in eye, anophthalmia and microphthalmia. FIG. 5 is a photograph of an anomalous 15-day-old embryo, whose male parent was irradiated with 3.0 Gy of nitrogen ion beams, showing excencephalia.

### Example 4

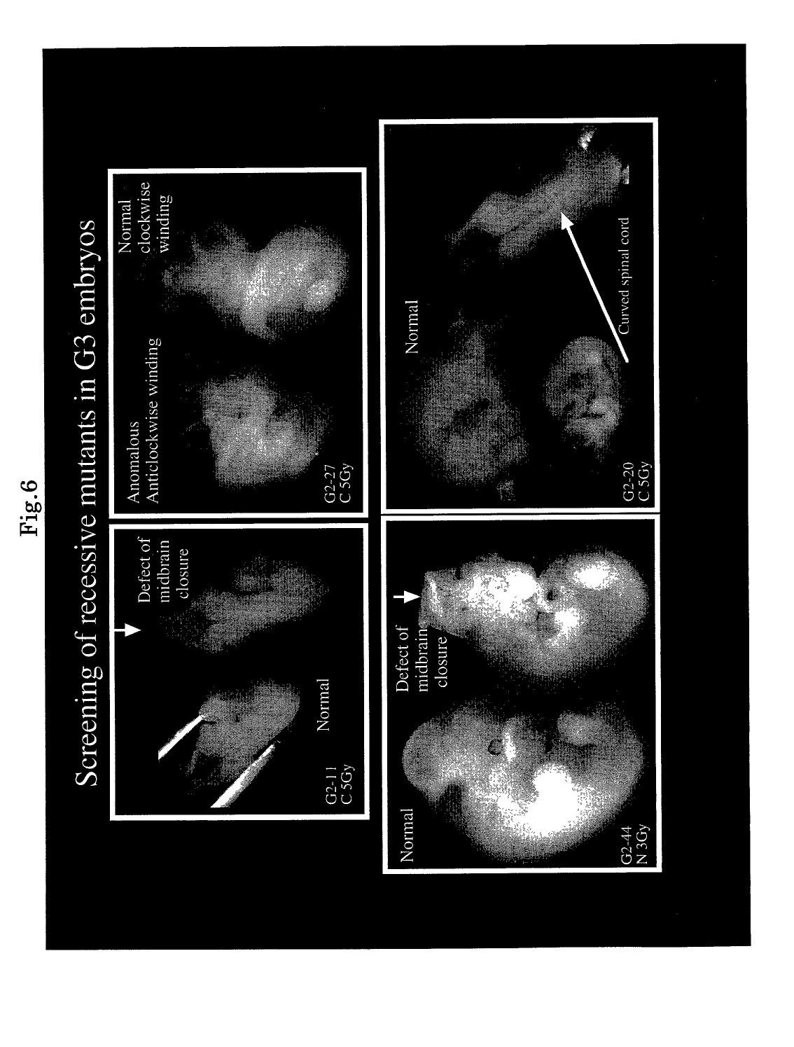

G1 mice produced by crossing of normal female mice with male mice treated with heavy ion beams were crossed with normal female mice to produce G2 mice. The G2 mice were backcrossed with the GI mice in order to observe the state of embryos (G3) produced in the pregnant female mice. As a result, implantation of 142 G3 embryos was confirmed in the female mice. Among these G3 embryos, 114 were normal, 21 showed some anomalies, and 7 were absorbed in the process of their growth. FIG. 6 shows photographs of the embryos with anomalies, wherein the embryos on left hand side show anomalies in brain, and the embryos on right hand side show anomalies in body axis (upper side) and spinal cord (lower side).

Advantageous Effect of the Invention

According to the present invention, animal mutants with various phenotypes or morphological characters can efficiently be created and can be utilized as a tool to analyze specific gene functions or as an animal model for studying human diseases.

All publications, patents and patent applications cited herein are incorporated herein by reference in their entirety.

What is claimed is:

1. A method for producing non-human animal mutants, characterized in that the method comprises irradiating a male germ cell with heavy ion beams and selecting a mutated animal from animals, which are produced from an egg and the irradiated male germ cell, or their generations.

2. The method of claim 1, wherein the male germ cell to be irradiated with heavy ion beams is a spermatozoon collected from a testis of a male mouse.

3. A non-human animal mutant produced by the method of claim 1 or claim 2.
